SEQUENCE LISTING (4 sequences)

SEQ ID NO: 1 - protein, 310 aa, Homo sapiens
MAFPKMRLMYICLLVLGALCLYFSMYSLNPFKEQSFVYKKDGNFLKLPDTDCRQTPPFLVLLVTSSHKQLAERMAIRQTWGKERMVKGKQLKTFFLLGTTSSAAETKEVDQESQRHGDIIQKDFLDVYYNLTLKTMMGIEWVHRFCPQAAFVMKTDSDMFINVDYLTELLLKKNRTTRFFTGFLKLNEFPIRQPFSKWFVSKSEYPWDRYPPFCSGTGYVFSGDVASQVYNVSKSVPYIKLEDVFVGLCLERLNIRLEELHSQPTFFPGGLRFSVCLFRRIVACHFIKPRTLLDYWQALENSRGEDCPPV

SEQ ID NO: 2 - protein, 331 aa, Mus musculus
MAPAVLTALPNRMSLRSLKWSLLLLSLLSFLVIWYLSLPHYNVIERVNWMYFYEYEPIYRQDFRFTLREHSNCSHQNPFLVILVTSRPSDVKARQAIRVTWGEKKSWWGYEVLTFFLLGQQAEREDKTLALSLEDEHVLYGDIIRQDFLDTYNNLTLKTIMAFRWVMEFCPNAKYIMKTDTDVFINTGNLVKYLLNLNHSEKFFTGYPLIDNYSYRGFFHKNHISYQEYPFKVFPPYCSGLGYIMSGDLVPRVYEMMSHVKPIKFEDVYVGICLNLLKVDIHIPEDTNLFFLYRIHLDVCQLRRVIAAHGFSSKEIITFWQVMLRNTTCHY

SEQ ID NO: 3 - protein, 324 aa, Drosophila melanogaster
MQSKHRKLLLRCLLVLPLILLVDYCGLLTHLHELNFERHFHYPLNDDTGSGSASSGLDKFAYLRVPSFTAEVPVDQPARLTMLIKSAVGNSRRREAIRRTWGYEGRFSDVHLRRVFLLGTAEDSEKDVAWESREHGDILQADFTDAYFNNTLKTMLGMRWASEQFNRSEFYLFVDDDYYVSAKNVLKFLGRGRQSHQPELLFAGHVFQTSPLRHKFSKWYVSLEEYPFDRWPPYVTAGAFILSQKALRQLYAASVHLPLFRFDDVYGIVALKAGISLQHCDDFRFHRPAYKGPDSYSSVIASHEFGDPEEMTRVWNECRSANYA

SEQ ID NO: 4 - protein, 322 aa, Caenorhabditis elegans
MFLCVRILKRKYHELSSFQKLLIFTITIFLLWVLGVVDKFRETSFGDFSWPLETRNLQLRSKFTKYPQCKFSGNGQKIIIIIIKSSAKNGPMRESVRKTWGVFRMIDGVEVMPIFIVGRVENMEIMRRIDVESEKYKDILAISDIDSYRNNTLKLFGAIDYAANPNQCSSPDFTFLVDDDYLVHIPNLVKFAKTKQKEELVYEGFVFDTSPFRLKIHKHSISLNEYPFSRYPPYVSAGAVFLTSETIARFRNSIRKLKMFPFDDVFTGILAKTVNVAATHNENFIFWCRRVSQKEWDDGVIAVHGYARKDLEYEYSQLNGFE

(19) United States
(12) Patent Application Publication
(10) Pub. No.: US 2003/0131378 A1
(43) Pub. Date: Jul. 10, 2003

(54) Methods for blocking resistance to bt toxins in insects and nematodes

(76) Inventor: Raffi V. Aroian, San Diego, CA (US)

Correspondence Address: BROWN, MARTIN, HALLER & MCCLAIN LLP, 1660 UNION STREET, SAN DIEGO, CA, 92101-2926, US

(21) Appl. No.: 10/344,440

(22) PCT Filed: Aug. 10, 2001
(86) PCT No.: PCT/US01/41687

(51) Int. Cl. A01H 1/00; C12N 15/82

(52) U.S. Cl. 800/279; 435/468

(57) ABSTRACT

Two genes involved in the resistance of insects to Bacillus thruingiensis toxins have been cloned providing an understanding of mechanisms of resistance to the toxins. Such an understanding allows for rational methods to modify or combine toxins to prevent or overcome Bt toxin resistance to improve crop protection.

### CROSS-REFERENCES TO RELATED APPLICATIONS

This application claims the benefit of priority of U.S. provisional application Serial No. 60/224,941 filed on Aug. 11, 2000 which is incorporated herein by reference in its entirety.

### GOVERNMENT INTEREST

The invention was made with government support from the National Science Foundation under grant number MCB-9983013.

### FIELD OF THE INVENTION

The invention relates to the genetics of mechanisms of resistance of insect crop pests to insecticides and the use of the knowledge of those mechanisms to prevent or circumvent pest resistance to improve crop protection.

### BACKGROUND OF THE INVENTION

The leading biorational pesticide Bacillus thruingiensis (Bt) is a ubiquitous gram-positive, spore forming bacterium that forms a parasporal crystal during the stationary phase of its growth cycle. Bt bacteria were identified as insect pathogens and their insecticidal activity was attributed largely or completely to the parasporal crystals encoded by the Cry genes, of which there are over 100 known isoforms. This observation led to the development of bioinsecticides based on Bt bacteria for the control of certain insect species among the orders Lepidoptera, Diptera, and Coleoptera. Further studies revealed isolates active against other insect orders (Hymenoptera, Homoptera, Orthoptera, and Mallophaga) and against nematodes, mites, and protozoa. Bt bacteria and toxins are useful alternatives or supplements to synthetic chemical pesticide application in commercial agriculture, forest management, and mosquito control.

Various strains of Bt bacteria are indigenous to many environments. Strains have been isolated worldwide from many habitats including soil, insects, stored product dust, and deciduous and coniferous leaves. The strains produce a wide variety of toxins due to a high level of genetic diversity and plasticity. Most Cry genes appear to reside on plasmids, autonomously replicating circular segments of DNA, often as parts of composite structures that include mobile genetic elements. Many of the toxin containing plasmids appear to be conjugative in nature, allowing for the transfer of Cry coding sequences between Bt bacterial strains.

Bt toxins are expressed during the stationary phase of growth of the bacteria and can account for 20-30% of the dry weight of the sporulated cell. Bt toxin proteins are toxic to insects during their larval stage. Their mechanism of action involves the solubilization of the protoxin crystals in the insect midgut, proteolytic processing of the protoxin by the midgut proteases, binding of the Bt toxin to the midgut receptors, and insertion of the toxin into the apical membrane to create ion channel pores, resulting in loss of membrane integrity, intestinal cell lysis, and insect death. Disruption of any of these steps can render the toxin inactive, making mechanisms of resistance difficult to predict.

The expression of Bt toxin genes only during the stationary phase of growth of Bt bacteria makes the use of the native organism for pesticidal control less desirable. Frequent reapplication of organisms that are non-trivial to produce is required. This problem was partially overcome by the transfer of Bt toxin genes into E. coli. The heterologous bacteria to expressed Bt toxins without exhibiting the growth phase limitations characteristic of the natural bacterial host species (Schnepf, U.S. Pat. No. 4,467,036). However, frequent reapplication was still required. Eventually, methods for the efficient transfer of genes into plants were developed (e.g. Donovan, U.S. Pat. Nos. 5,187,091; Adang, 5,380,831; Fischhof, 5,500,365), and the Bt toxin genes were transferred into plants for continuous expression.

The use of Bt toxins in agriculture is widespread. In 1999, approximately 35% of corn, 30% of cotton, and 4% of potatoes were produced using transgenic plants expressing Bt toxins. A number of other Bt expressing crops are coming into use including asparagus, broccoli, carrots, cucumbers, alfalfa, soybeans, apples, peas, and lotus. The use of transgenic plants reduces the need for insecticide spraying resulting in a lower environmental impact.

As the use of any toxin for pest control spreads, the resistance of the pests to the toxin will spread. Resistance of diamond back moths to Bt toxins Cry1A, 1B and 1C has been documented in the field. Many other resistances have been observed in the laboratory. Theories on mechanisms of Bt toxin resistance have been proposed, however, no Bt toxin resistance genes have been identified and the mechanisms of resistance are unknown. This is partially due to a lack of detailed information on agricultural pests. Little is known about the genetics of agricultural pests and methods for studying them are not well established. This increases the difficulty of understanding the process of Bt toxin resistance.

Multiple steps are required for the activation of Bt toxins; therefore, many mechanisms by which pests could evade the toxin exist. These include altered gut pH to decrease solubilization, under- or overproteolysis of the toxin, changes in the receptors on the surface of the midgut, changes in the secondary modifications of the toxin receptors, hindered pore formation or the plugging of pores, increased rate of epithelium repair, and toxin recognition resulting in decreased consumption of toxic plants. Different Bt toxins are effective against different species of insects, suggesting that there are differences in their mechanisms of action. For this reason, it seems logical that inserting multiple Bt toxin genes into a single plant, or growing plants expressing different Bt toxins in the same field, could be an ideal method for overcoming pest resistance. However, with no knowledge of the mechanisms of resistance, it is not possible to group toxins in a rational manner. Exposure of an insect to a single Bt toxin can result in resistance to multiple isoforms of the toxin, therefore arbitrarily combining toxins would not be useful in overcoming toxin resistance. Similarly, there is no way to rationally modify individual toxins to circumvent resistances.

Modified Bt toxins have been developed to increase their activity and broaden their host range. English, et al. (U.S. Pat. No. 6,063,597) teach the use of a variety of mutated Cry3B proteins and protein fragments, containing one or more point mutations, for use as insecticides with Coleopteran insects. Sivasubramanian, et al. (U.S. Pat. No. 5,306,628) teach the creation of a hybrid toxin, containing an insect midgut binding motif from a virus or glycoprotein fused to a Bt toxin to increase the host range of a toxin. The modified toxins provided by these inventions may be useful in overcoming some resistances that develop in insect populations; however, they do not teach a method for selecting the best toxin, or combination of toxins, to overcome toxin resistance.

### SUMMARY OF THE INVENTION

The invention is a method for the protection of crops comprising the rational modification, combination or supplementation of Bt toxins for the control of pests. Understanding mechanisms of resistance allows rational choices to be made regarding the use of Bt toxins to prevent the development of pest resistance or to overcome existing pest resistance to Bt toxins.

The invention is the cloning of genes responsible for the resistance to the Bt toxin Cry5B by a genetic screen using the model organism C. elegans. In the screen animals were mutagenized and selected for their ability to grow on E. coli, their normal food source, expressing the Bt toxin Cry5B. The mutant animals were found to fall into five complementation groups and were named bre mutants for Bacillus toxin resistance mutants. Further analysis of the genes responsible for toxin resistance revealed that two of the genes, bre-3 and bre-5 have significant homology to known Drosophila genes egghead and brainiac, which are known to function coordinately in the same signaling pathway. The discovery of the role of widely expressed genes in Bt resistance demonstrates the commonality of resistance mechanisms and the utility of the model system.

The invention is a method to rationally overcome resistances to Bt toxins. This can be accomplished by direct modification of Bt genes and by combination of Bt toxins with other compounds, including other Bt toxins, for the killing of resistant pests and to enhance crop protection. For example, inhibition of glycosylation of Bt toxin receptors in the insect midgut results in toxin resistance due to decreased toxin binding. Therefore, one can overcome the resistance by the addition of a non-glycosylation dependent gut binding motif to the toxin. Using a standard molecular biology techniques, the coding sequence for an insect gut binding motif can be added. Binding of the toxin to the gut can be mediated by protein, lipid, or carbohydrate domains.

Insects may become cross-resistant to a number of Bt toxins after having been exposed to only a single toxin. The identification of mechanisms of resistance to Bt toxins can provide a method for the rational stacking of toxins in plants such that the mechanisms of resistance to the toxins are non-overlapping. The insertion of genes into plants is non-trivial, and the space and time required for the growth of plants limits their use in a high throughput assay. Genes can easily be inserted into E. coli that can be used in a high throughput screen to test the effectiveness of combinations of toxins, and the ability of the animals to develop resistance to a combination of toxins. Using the screen, one can readily identify Bt toxins that bind to the midgut via different carbohydrate modifications. Such toxins can be used in combination with each other in crops as downregulation of two glycosylation or signaling pathways in the insect would likely decrease the fitness of the insect, such that resistance to the two toxins would be disadvantageous.

Resistance to Bt toxins can result from modification of glycosylation pathways. Major changes in glycosylation pathways can result in a new susceptibility in the resistant insects that could be exploited. For example, a brief dose of a glycosylation inhibitor would not be toxic to most organisms. A single glycosylation inhibitor would not inhibit all glycosylation pathways; therefore, most animals would be able to compensate for disruption of a single pathway. However, an organism that has downregulated or eliminated a glycosylation pathway would be more susceptible to treatment with a glycosylation inhibitor.

The invention is a method to develop regimens for level and frequency of dosing of toxins to inhibit the development of resistance. Toxins can be constitutively co-expressed in plants. Alternatively, one toxin can be expressed by the plant, and the other can be added by spraying or other periodic application or expression method to increase killing of resistant pests without increasing resistance in non-resistant pests. Toxins can be placed under the control of different promoters, either constitutive or inducible, to vary the level and frequency of the toxins expressed.

The invention is the use of the nematode C. elegans as a model for Bt toxin resistance in agricultural pests. The identification of genes common to a number species of insects as Bt toxin resistance genes demonstrates the utility of C. elegans in understanding general mechanisms of resistance. In the assay, the animals are subject to random chemical mutagenesis and selected for resistance to Bt toxins expressed in E. coli, the usual food source of the nematodes. Resistant animals are isolated into individual cultures where they reproduce hermaphroditically. Resistance genes are cloned by complementation and analyzed for function by a number of well established methods. C. elegans can also be used to understand the development of toxin resistance and mechanisms of cross-resistance.

### BRIEF DESCRIPTION OF THE DRAWINGS

The present invention will be better understood from the following detailed description of an exemplary embodiment of the invention, taken in conjunction with the accompanying drawings:

FIG. 1. BRE-5 encodes a putative galactosyltransferase that is required in the C. elegans gut for Bt toxin action. The sequences are a CLUSTALW (version 1.81) alignment of BRE-5 protein with human b1,3-galactosyltransferase polypeptide 5 (hB3T5); mouse b1,3-galactosyltransferase polypeptide 3 (mB3T3); and Drosophilia BRAINIAC (Brn). The putative transmembrane domain is underlined. The DXD and DVFTG motifs are double underlined. The location of the two arginines mutated in the bre-5 alleles are indicated. ye107 alters an arginine conserved in all b1,3-galactosyltransferases; ye17 introduces a stop codon upstream of the conserved (E/D)DV galactosyltransferase motif.

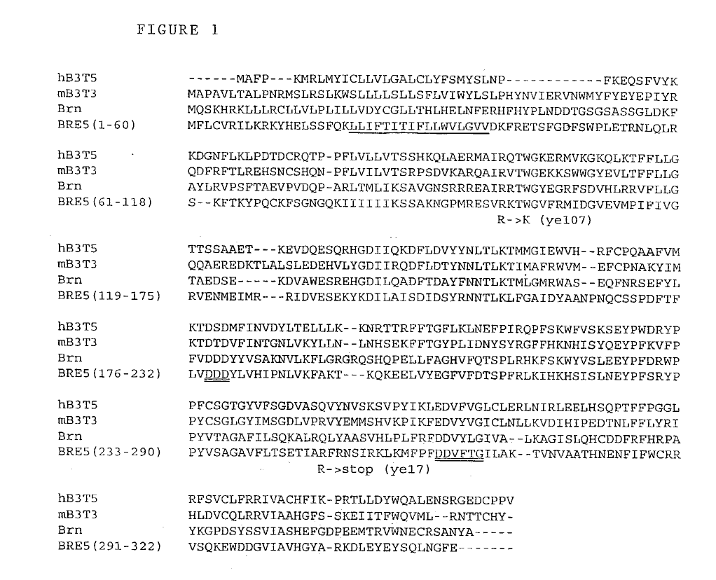

### DETAILED DESCRIPTION AND PREFERRED EMBODIMENTS

One of the biggest hurdles in developing effective methods to overcome insect resistance to Bt toxins is a lack of understanding of the mechanisms of resistance. This invention is the cloning of the first two genes involved in resistance of insects to Bt toxins. The genes were cloned using the model organism C. elegans in a genetic screen. C. elegans is a nematode that has been used as a genetic model to analyze a number of biological processes. Libraries of mutant animals can be easily generated and subjected to screening methods to isolate the characteristics of choice. C. elegans are hermaphrodites which facilitates the establishment and maintenance of isogenic strains. The generation time of C. elegans is short (3.5 days at 20° C.) and 200-300 progeny are produced per generation. The genome has been completely sequenced and studies have clustered genes into functional groups. Genetic maps and techniques are well established.

A high throughput genetic screen was established to identify genes that are involved in resistance to Bt toxins. C. elegans were grown on E. coli, their standard food source, and subjected to mutagenesis by EMS. Animals were allowed to self for two generations before being transferred onto plates of E. coli expressing Cry5B, or into individual wells of 96 well plates containing Cry5B. Survivors were isolated from mixed plates. Individual strains were expanded for further analysis.

Using the above method, over 90,000 mutagenized F2 animals were screened, and over 40 mutants were isolated in the first round. Linkage mapping and subsequent three factor crosses were used to locate the mutations in the genome. The mutant animals were found to fall into five complementation groups. Cosmids carrying approximately 40 kb of C. elegans sequence were injected to complement the mutations to confer sensitivity to Cry5B. Fragments from complementing cosmids were injected to identify the mutated gene that conferred resistance. Resistance genes were amplified from the animals by PCR and sequenced using an automated sequencer.

bre mutants were mapped to different chromosomes in the C. elegans, with bre-1 and bre-5 on LGIV, bre-2 and bre-3 on LGIII, and bre-4 on LGI. All were found to be recessive mutations.

bre-3 was cloned and found to be the open reading frame B0464.4 as defined by the C. elegans sequencing project. There was no other information regarding this gene or gene product of C. elegans. BRE-3 was found to be 60% identical to Drosophila Egghead at the amino acid level. Although the function of Egghead/BRE-3 is not known, hydropathy analysis has revealed the presence of at least 4, possibly 5, transmembrane domains. Studies on Egghead in Drosophila indicate that it functions in a signaling pathway with the Brainiac, most likely as a sugar transporter or a facilitator for Brainiac carbohydrate modification.

bre-5 mutants were complemented by a previously unidentified open reading frame on the cosmid T12G3 (C. elegans genome center) which was not predicted by the C. elegans sequencing project. BRE-5 was found to be 35% identical to Drosophila Brainiac at the amino acid level and to contain all of the motifs characteristic of beta 1,3-galactosyltransferases.

Based on the identity of BRE-5 as a putative beta 1,3-galactosyltransferase, and the fact that Egghead and Brainiac in Drosophila function in the same signalling pathway suggests that the reduction or elimination of certain carbohydrate modifications plays an important role in the development of resistance to Bt toxins. This conclusion is supported by in vitro Bt toxin gut binding assays in which GalNac was able to specifically inhibit the binding of Bt toxin Cry1AC to insect midgut.

bre-5 mutants were tested for cross resistance to Cry14A and Cry21. They were not found to be fully cross resistant to either toxin, suggesting that the toxins bind to the midgut via different receptors. More interestingly though, bre-5 mutants were found to be resistant to a low level of Cry14A and sensitive to a high level of Cry14A. This indicates the presence of multiple binding sites in the midgut for Cry 14A, a high affinity binding site that requires a GalNac carbohydrate modification, and a low affinity binding site that does not require a GalNac modification. Such studies present a mechanism for the presence of resistance to multiple Bt toxins after exposure to only one toxin. Moreover they reveal the presence of alternate binding sites that would not likely be found by any other method.

This pattern of cross resistance was somewhat surprising as Cry21A is more similar to Cry5B than Cry 14A. Recently, the structures of three Bt toxins, Cry1Aa, Cry2Aa and Cry3A, have been determined. Although the overall identity of the proteins is as low as 17%, they all contain a common structural element, the β-prism. This unusual structure has been seen previously only in two plant lecithins. β-prisms bind carbohydrates, specifically Gal-β-1,3-GalNac. This common structural feature of the Bt toxins is likely one of the mediators of gut binding of the toxins and provides a rational explanation for the cross-resistance seen in insects to Bt toxins with overall low homology. The presence of a distinct gut binding region provides a rational site for modification of the Bt toxins to overcome resistance. The region could either be subjected to random mutagenesis to modify the specificity of the binding of the domain. Such screening could be performed using any of a number of library screening methods including phage display or affinity chromatography using carbohydrates other than the natural ligand as a probe. Alternatively as the binding domain is a modular unit, it could be removed and replaced by a different gut binding domain not dependent on glycosylation without altering the function of the remainder of the toxin.

A hypothesis on insect toxin resistance proposed mutations in secondary protein modification pathways as a possible mechanism, but suggested that mutations in such pathways would cause a reduction in fitness in animals in exchange for toxin resistance. A reduction in brood size was seen in some of the bre mutants, however, no major abnormalities were observed. Due to the method of the screen, resistance mechanisms that resulted in a significant reduction in fitness would not be selected, analogous to the selection process in nature. However, it is unlikely that no fitness cost is incurred as a result of an alteration in a secondary protein modification pathway, resulting in a weakness in the organism that can be exploited. Such weaknesses can not be exploited without the identification of the mechanisms involved in toxin resistance.

### EXAMPLE 1

Addition of midgut binding motifs to Bt toxins. Hybrid toxins expressing Cry genes fused to a gut binding motif can be used to circumvent resistances due to changes in glycosylation pathways. This can be accomplished by addition of a number of motifs including sites for lipid modification (e.g. prenylation sites), multiple tandem carbohydrate modification sites (e.g. glycosylation sites), or protein motifs (e.g. midgut binding motifs from different Bt toxins that bind to different carbohydrates, proteins that bind to structural proteins of the insect gut). Coding sequences for such motifs could be readily incorporated into the coding sequence for the Bt toxin and inserted into plants by standard methods. This would abrogate the need for specific carbohydrate modifications of receptors in the gut eliminating one option for Bt toxin resistance.

### EXAMPLE 2

Random mutagenesis of toxins to overcome resistance. Less directed methods of modification of Bt toxins can be used to overcome resistance to a toxin. For example, Cry5B can be subjected to random mutagenesis by any of a number of methods including error prone PCR mutagenesis. Primers with endonuclease restriction sites that anneal to the ends or internal sequences of Cry5B can be designed. PCR products are digested, ligated into an appropriate vector, and transformed into E. coli for expression. Alternatively, a pool of candidates for screening could be generated by the protein evolution methods of Minshull and Stemmer (Protein evolution and molecular breeding. Curr. Opin. Chem. Biol. 3:284-90.1999; incorporated herein by reference). Individual colonies expressing mutant Cry5B are cultured as individual clones and transferred to plates for use as a food source for bre animals. Mutant cry5B clones capable of killing bre animals are sequenced. Thus, mutations in Cry5B that are able to kill resistant animals can be identified. Such a toxin can be used alone or stacked with wild type Cry5B to prevent or overcome pest resistance.

### EXAMPLE 3

Exploiting the development of resistance pathways. Insects that have downregulated one glycosylation pathway are more sensitive to a low concentration of glycosylation inhibitor that is not sufficient to harm plants or other non-resistant animals. A number of glycosylation inhibitors are expressed in the seeds of leguminous plants. They include indolizidines alkaloids (swainsonine [SWS] and castanospermine [CS]), polyhydroxylated pyrrolidines and piperidines (N-methyideoxynojirimycin [MdN] and 1-deoxymannojirimycin [DMM]), and myoinositol derivatives. The purified compounds are commercially available, but crude preparations would be sufficient for use in agriculture. Such compounds can be applied to plants, either on a constant or intermittent basis to kill pests that have developed resistance to Bt toxins by downregulating glycosylation pathways.

### EXAMPLE 4

Synthetic lethal screen to determine rational combinations of toxins. The concept of “synthetic lethal” mutations is well established in genetics. Two independent mutations are tolerated by an organism, but the combination of two mutations in a single organism results in death. C. elegans strains that demonstrate no cross resistance can be mated to identify synthetic lethal combinations of toxin resistances. The toxins can be co-expressed in plants as the development of resistance to both toxins would lead to death of the animal.

### EXAMPLE 5

Cross resistance screen. C. elegans mutants resistant to one Bt toxin can be tested for innate resistance to other Bt toxins by growing them on E. coli expressing other Bt toxins. The toxins can be expressed constantly at a low or high level or intermittently depending on the promotor driving the expression of the toxin. For low level expression of toxin, a mixed population of bacteria can be used such that only a portion of the bacteria express the Bt toxins of interest. Such promoter systems are well known to those skilled in the art.

The ability of C. elegans to develop cross resistance to a second toxin can be tested by a screen similar to that used to identify the bre mutants. For example, bre-3 animals are be subjected to mutagenesis by EMS and allowed to self for two generations on E. coli expressing Cry5B to eliminate animals that have become resensitized to Cry5B in the process of mutagenesis. Animals are transferred to E. coli expressing a Cry protein to which they have no innate cross-resistance as determined by the above assay (e.g. Cry 1A). If resistant animals are found at a high frequency, resistances would likely develop rapidly in the wild. If upon repeated rounds of screening no doubly resistant animals are found, it is likely that the combination of resistances is lethal and can be useful in an agricultural setting.

### EXAMPLE 6

Identification of multiple binding sites for Bt toxin in the gut. bre-5 animals were tested for cross resistance to other Cry proteins by growth on E. coli expressing various toxins. bre-5 animals are resistant to a low level of Cry14, however, they are sensitive to a high level of Cry14. This indicates that there are two receptors for Cry14 on the brush border membrane. The high affinity receptor requires specific β-1,3-GalNac modification to bind the toxin, but the low affinity receptor does not. There is no suggestion in the prior art for the presence of multiple receptors with different affinities. Such a discovery suggests a method of pest control involving the intermittent application of high doses of a second toxin in combination with a toxin expressed in plants. The second, high dose toxin could be applied directly to plants or it can be placed under the control of an inducible promotor. The inducing factor can be applied to the plants for intermittent expression. A modified version of the screen could be used to determine the best frequencies for application of the secondary toxin for maximum killing of pests with the lowest frequency of the development of multiple toxin resistance.

### EXAMPLE 7

Identification of essential genes involved in Bt toxin resistance. It is likely that Bt toxins have evolved mechanisms that act through essential host genes. A screen that uses survival as the endpoint may fail to uncover resistance genes that are also important for host viability and fertility which may be mutated in resistant pest populations. A similar screen for essential genes that can produce resistance to toxins can be preformed on L4 (juvenile) animals that are homozygous (F2 generation) for temperature sensitive mutations. Mutations in essential genes are often tolerated if the shift to the non-permissive temperature occurs after the completion of development. Homozygous animals are grown at the permissive temperature until the L4 stage and then switched to the non-permissive temperature, inactivating a toxicity-mediating protein. The animals are then transferred to plates containing E. coli expressing a Bt toxin, and resistant animals are recovered and maintained at the permissive temperature. Progeny (F3 generation) of these animals are tested for temperature sensitivity with regard to viability or fertility. They are then tested for the linkage of this defect with the resistance phenotype. Such an assay allows for the identification of essential genes that cannot be detected by conventional screening. Thus, Bt toxins for which no resistant animals can be found by the screen used to identify bre-3 and bre-5 mutations can be tested in this assay to determine by what novel mechanisms of resistance can develop. Understanding the trade-offs between resistance and host fitness would allow the prediction of which resistant loci are most likely to change, and which steps in toxin action are most susceptible to host-mediated inactivation.

Although an exemplary embodiment of the invention has been described above by way of example only, it will be understood by those skilled in the field that modifications may be made to the disclosed embodiment without departing from the scope of the invention, which is defined by the appended claims.

I claim:

1. A method for protection of crops comprising application to crops or expression in crops of Bt toxins containing modifications to facilitate binding of the modified Bt toxins to insect gut.

2. A method as in claim 1, wherein the modifications comprise addition of lipid modification sites.

3. A method as in claim 1, wherein the modifications comprise addition of carbohydrate modification sites.

4. A method as in claim 1, wherein the modifications comprise addition of protein fragments that bind structural or surface elements of the insect gut.

5. A method as in claim 1, wherein the modifications comprise addition of receptor binding domains from different Bt toxins.

6. A method as in claim 1, wherein the modifications comprise mutations introduced by random or directed mutagenesis.

7. A method for protection of crops comprising application or expression of a plurality of Bt toxins with non-overlapping mechanisms of resistance.

8. A method as in claim 7, wherein the plurality of Bt toxins are expressed in a single plant.

9. A method as in claim 7, wherein at least one of the plurality of Bt toxins is expressed in plants and another of the plurality of Bt toxins is applied exogenously.

10. A method as in claim 7, wherein a plurality Bt toxins are all used at consistent levels.

11. A method as in claim 7, wherein at least one of the plurality of Bt toxins is expressed continuously and at least another of the plurality of Bt toxins is expressed intermittently.

12. A method as in claim 7, wherein the plurality of Bt toxins are expressed by different plants in the same field.

13. A method for protection of crops comprising application or expression of at least one Bt toxin is supplemented with a non-Bt toxin.

14. A method as in claim 13, wherein the supplement comprises glycosylation inhibitors.

15. A method as in claim 13, wherein the supplements are applied to plants intermittently.

16. A method for identification of mechanisms of resistance in agricultural pests comprising C. elegans nematodes as a model system for insecticide resistance.
